(19) United States
(12) Patent Application Publication
(10) Pub. No.: US 2025/0134617 A1
(43) Pub. Date: May 1, 2025

(54) MIGS ASSIST

(71) Applicant: Diane Spizzirro, Larchmont, NY (US)

(72) Inventor: Diane Spizzirro, Larchmont, NY (US)

(21) Appl. No.: 18/496,855

(22) Filed: Oct. 28, 2023

(51) Int. Cl. A61B 90/20; A61B 3/13

(52) U.S. Cl. CPC A61B 90/20; A61B 3/13

(57) ABSTRACT

A retrofit handle for use with a surgical ophthalmic microscope. The handle is configured to replace or attach to an existing adjustment knob and is used to adjust the microscope without requiring the surgical field to be broken to use the existing adjustment knob. The handle is also designed to permit multiple complete rotations without requiring the hand of a user to be removed from the handle and then grasp it again.

### BACKGROUND

In order to perform eye surgery, physicians use an ophthalmic microscope, also called a surgical microscope, to see the very small structures within the globe of the eye. The surgical microscope is held over the patient's head with a stand, while the patient is lying flat on a stretcher. The microscope is moved into position immediately above the eye, and the surgeon looks through the lenses, called oculars, for most surgeries of the eye.

As a general rule, all ophthalmic surgical microscopes have a knob on the side arm of the head of the microscope that allows the surgeon or staff to angle the microscope appropriately for the surgeon's preference. The knob itself tends to be small and not ideal for the task of tilting the head of the microscope. There are procedures that require the surgeon angle the microscope, instead of maintaining it in a purely vertical position, in order to see small structures within the eye that are difficult or impossible to see if the microscope is not angled appropriately. For example, there is a procedure for treating glaucoma in which a drainage device is placed in the trebecular meshwork of the eye. This procedure is generally referred to as Micro Invasive Glaucoma Surgery, or MIGS. Because the trebecular meshwork is in the anterior chamber angle of the eye, the surgical microscope must be tilted to nearly 45 degrees off of vertical while a prism is held on the cornea in order to perform the MIGS procedure.

Virtually every existing ophthalmic surgical microscope uses a knob to angle the head of the microscope that is poorly designed. In order to angle the head of the surgical microscope correctly, the knob often must be turned 20-30 times. Additionally, immediately after the MIGS procedure the microscope head must be returned to the original, vertical position, requiring just as many turns back; this is more difficult because of the weight of the head of the microscope. This is time consuming and uncomfortable, and the knob must have a sterilized cap covering it during surgery to keep the surgeon and nurse from accidentally touching a non-sterile portion of the scope; the extensive hand movement required to turn the knob can result in accidentally breaking sterility. Although knobs are a very poor design for this purpose, all existing ophthalmic surgical microscopes on the market use them.

This flaw is known, and other solutions are available to address it. The most common is a motorized tilt mechanism that can be attached to the microscope. However, this is both expensive and bulky, with the bulk being inconveniently added to the microscope head.

### SUMMARY

A replacement handle, retrofittable onto any existing ophthalmic surgical microscope, which gives more control by using a pivoting two point handle instead of a simple knob. The addition of an arm between the point about which the original knob rotates and the point of action by the user permits a reduction in the number of adjustments by the user required to get the angle of the surgical microscope head to the desired angle. The “two point” aspect of the pivoting two point handle permits the user to grasp the handle once and continue to rotate the handle until the desired angle is reached, instead of repeatedly releasing and grabbing the handle again, similar to an old manual car window handle. This continuous adjustment option reduces the likelihood of breaking sterility, because every instance of releasing and grasping an adjustment knob is another opportunity to accidentally break sterility.

### DETAILED DESCRIPTION

### Brief Description of Figures

FIG. 1: inside perspective view of handle, first embodiment

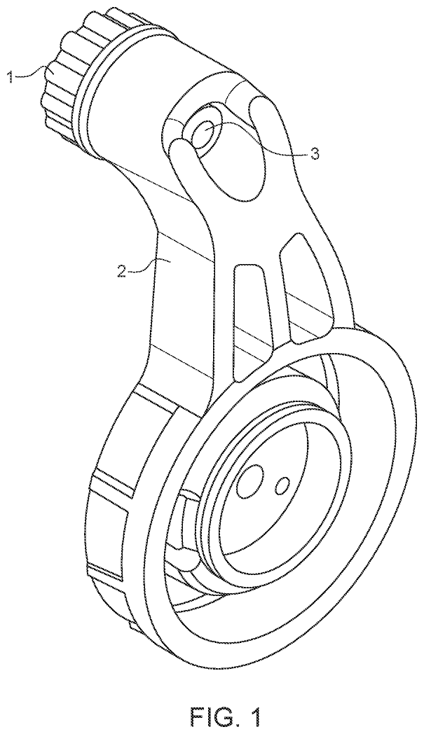

FIG. 2: outside perspective view of handle, first embodiment

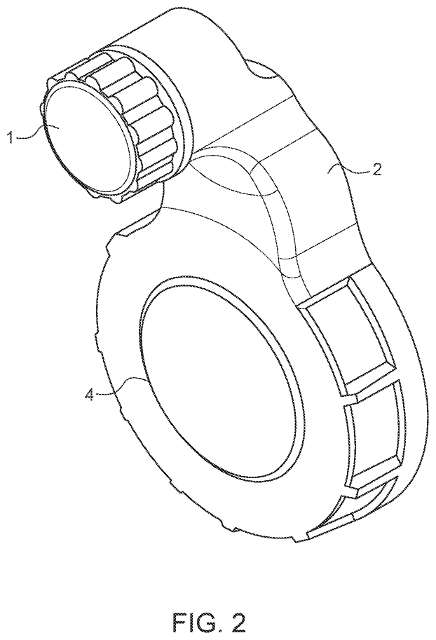

FIG. 3: cross section of the first embodiment of the handle taken across the line of symmetry of the handle

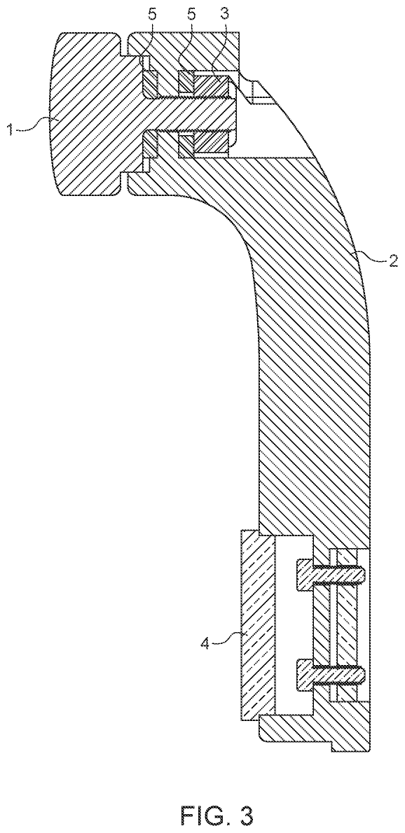

FIG. 4: inside perspective view of handle, second embodiment

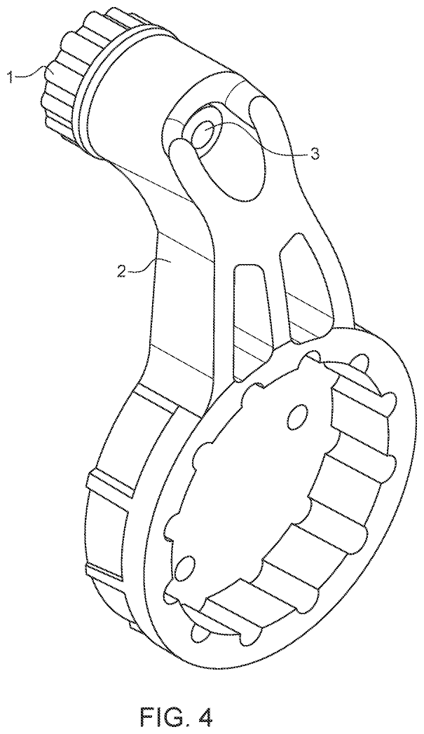

FIG. 5: outside perspective view of handle, second embodiment

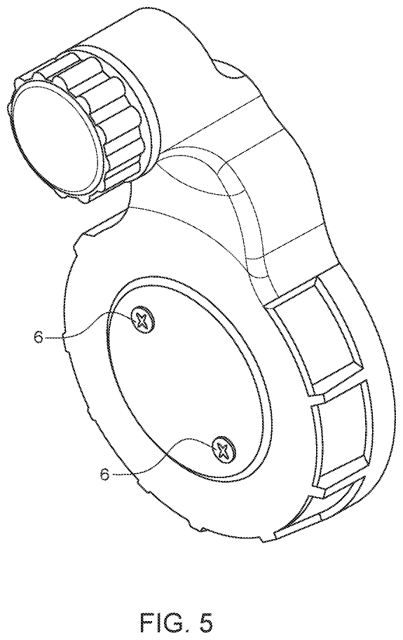

In the following description, like reference numbers are used to identify like elements. Furthermore, the drawings are intended to illustrate major features of the exemplary embodiments in a diagrammatic manner. The drawings are not intended to depict every feature of every implementation and are not drawn to scale.

In the following description, numerous specific details are set forth to clearly describe the embodiments disclosed herein. However, one skilled in the art will understand that some well known features have not been described in detail so as not to obscure the invention. In particular, the microscope itself is not the focus of the invention, and therefore has not been addressed.

In the first exemplary embodiment, the retrofittable handle includes the knob (1), a washer (5), a nut (3), an arm body (2), and a cover (4). The retrofittable handle is configured such that the knob may be continuously held while rotating the handle any amount, with the knob of the handle rotating freely relative to the arm. The cover (4) and arm body (2) are dimensioned so as to permit the retrofittable handle to replace an existing adjustment knob, with the cover being circular and centered over the center of rotation of the knob being replaced.

Because the structures of the surgical microscopes being modified by the retrofittable handle vary, the specifics of the connection between the handle and the machine will vary, and the washer and nut may not be necessary in all cases. Simultaneously, a different structure to connect the retrofittable handle to the surgical microscope may be necessary in other cases to ensure a clean, non-slipping connection. Therefore, the washer and nut are considered to be means for enabling the knob to rotate freely relative to the arm such that the angle of the head of the microscope can be adjusted with a single grasping of the handle regardless of the angle to which the microscope is being adjusted.

In the second exemplary embodiment, the retrofittable handle goes over the existing knob, replacing it without removal. Therefore, the original equipment manufacturer's structure that enables the knob to turn, in combination with how the retrofittable handle is attached to the existing knob, forms the equivalent means for enabling the knob (1) to rotate freely relative to the arm (2), with the connection of the cover (4) to the existing knob letting the retrofittable handle turn the existing knob such that the angle of the head of the microscope can be adjusted with a single grasping of the handle regardless of the angle to which the microscope is being adjusted. The cover may be attached to the existing knob via a set screw or screws (6).

While illustrative embodiments of the invention have been shown and described, variations and alternative embodiments will occur to those skilled in the art. Such variations and alternative embodiments can be made without departing from the scope of the invention as defined in the claims.

As used in this specification and the appended claims, the singular forms “a” and “an” indicate a single element, while “the” may refer back to single or plural referents. Unless defined otherwise, all technical and scientific terms used herein have the same meaning as commonly understood by one of ordinary skill in the art to which the disclosure pertains.

The above detailed description of exemplary and preferred embodiments is presented for the purposes of illustration and disclosure in accordance with the requirements of the law. It is intended to be exemplary but not exhaustive, and is not intended to limit the invention to the precise forms described, but only to enable others skilled in the art to understand how the invention may be suited for a particular use of implementation. No limitation is intended by the description of exemplary embodiments which may have included tolerances, feature dimensions, specific operating conditions, engineering specifications, or the like, and which may vary between implementations or with changes to the state of the art, and no such limitation should be implied therefrom. Applicant has made this disclosure with respect to the current state of the art, but also contemplates advancements and that adaptations in the future may take into consideration those advancements in accordance with the then current state of the art. It is intended that the scope of the invention be defined by the Claims as written and equivalents as applicable. Reference to a claim element in the singular is not intended to mean “one and only one” unless explicitly so stated. No claim element herein is intended to be construed under the provisions of 35 U.S.C. 112(f), unless the element is expressly recited using the exact phrase “means for . . . ” and no method or process step herein is to be construed under the provisions of 35 U.S.C. section 112(f) unless the step, or steps, are expressly recited using the exact phrase “step(s) for . . . ”.

1. A retrofittable handle for a surgical microscope comprising a knob, an arm, and a cover, wherein the retrofittable handle is configured to replace the knob for adjusting the angle of the head of the surgical microscope originally provided by the manufacturer of the surgical microscope.

2. The retrofittable handle for a surgical microscope of claim 1, further comprising a means for enabling the knob to rotate freely relative to the arm such that the angle of the head of the microscope can be adjusted with a single grasping of the handle regardless of the angle to which the microscope is being adjusted.

3. The retrofittable handle for a surgical microscope of claim 1, wherein the retrofittable handle is configured to replace the knob for adjusting the angle of the head of the surgical microscope originally provided by the manufacturer of the surgical microscope after removal of the knob, and the means for enabling the knob to rotate freely relative to the arm such that the angle of the head of the microscope can be adjusted with a single grasping of the handle regardless of the angle to which the microscope is being adjusted comprises a washer and a nut.

4. The retrofittable handle for a surgical microscope of claim 1, wherein the retrofittable handle is configured to cover and therefore replace the knob for adjusting the angle of the head of the surgical microscope originally provided by the manufacturer of the surgical microscope.

5. The retrofittable handle for a surgical microscope of claim 4, further comprising a set screw wherein the set screw and retrofittable handle are arranged to use the set screw to fasten the retrofittable handle to the knob it is covering.
